(19) United States
(12) Patent Application Publication
(10) Pub. No.: US 2023/0285704 A1
(43) Pub. Date: Sep. 14, 2023

(54) AEROSOL HIGH FLOW THERAPY APPARATUS

(71) Applicant: STAMFORD DEVICES LIMITED, GALWAY (IE)

(72) Inventors: John POWER, Galway (IE); Aidan DUFFY, Galway (IE); Micheal CASEY, Galway (IE)

(73) Assignee: STAMFORD DEVICES LIMITED, GALWAY (IE)

(21) Appl. No.: 18/040,163

(22) PCT Filed: Aug. 20, 2021
(86) PCT No.: PCT/EP2021/073209

§ 371 Date: Feb. 1, 2023

(30) Foreign Application Priority Data
Sep. 1, 2020 (EP) 20193976.6

(51) Int. Cl. A61M 16/06; A61M 16/16; A61M 16/00; A61M 16/10; A61M 16/20

(52) U.S. Cl. CPC A61M 16/0677; A61M 16/0622; A61M 16/16; A61M 16/0096; A61M 16/1075; A61M 16/1005; A61M 16/022; A61M 16/1055; A61M 16/0093; A61M 16/20; A61M 2016/0027; A61M 2205/3334; A61M 2205/3337

(57) ABSTRACT

A patient interface (1) is for aerosol treatment, having a base (2) to surround the patients mouth and nose and engage the skin with a resilient seal, and with a strap (8) to attach to a patients head. There is a support (3) on and across the base for supporting an aerosol delivery head with prongs (4, 5). An enclosed volume is formed in the interface by attachment of a shell (10), which has an extraction port (11) for attachment of an extraction system (20) to extract gas from this volume. An HFNT system includes a patient interface surrounding the nose and mouth and an aerosol delivery apparatus (4, 6), an extraction apparatus (20), and a controller (100) to control delivery of aerosol and/or gas to the interface and to extract gases from a volume enclosed by the interface. Because of the fully sealed volume within the interface there are a wide range of control scenarios possible, using pressure sensing in the volume, bypass valves (201), dynamically-controllable nebulizer (203).

### INTRODUCTION

The present invention relates to aerosol therapy, especially high flow therapy.

High flow nasal therapy (HFNT) will typically deliver an air/oxygen/aerosol mix to a patient at a rate that exceeds their peak inspiratory rate. An example is 50 LPM treatment vs. an average inhalation of about 20 LPM (averaged across the inhalation period of a breath) with a peak inhalation of about 35 LPM. Aerosol delivered into the high flow stream will be homogeneously distributed. Therefore, the excess airflow contains aerosolised drug that the patient cannot absorb and this results in reduced efficiency. Also, this excess will disperse into the surrounding room air. This is a fugitive emission that potentially exposes clinicians, patients and visitors to aerosolised drugs and patient-generated pathogens.

Also, during exhalation (typically through the mouth), the high flow therapy may continue to deliver to the nasal cavity. A portion of this flow will travel into the cavity and exit the patient's mouth, and this flow augments the exhalation flowrate and has the potential to collect patient pathogens. The remainder of the flow will exit the cavity via the nostrils, but prior to this it also can collect pathogens.

WO2015/155342A (Stamford Devices Ltd) and WO2019/007950A (Stamford Devices Ltd) describe HFNT systems, in which aerosol is delivered primarily during reduced gas flow periods, in order to increase efficiency and reduce losses. US2012/285455 (Varga et al) describes a mask for patient ventilation. US2004/244799 (Landis) describes a tube seal adapter for face masks. WO2018/204969 (P & M Hebbard PTY) describes a sealing pad for a respiratory mask. WO2019/159063 (Fisher & Paykel) describes a mask which is fitted over a nasal prong interface

The invention addresses the problem of achieving effective aerosol treatment with reduction or elimination of gas losses, particularly for high flow treatment.

### SUMMARY OF THE DISCLOSURE

We describe a patient interface for aerosol treatment, the interface comprising a base configured to surround at least part of a patient's mouth and nose and engage the skin with a resilient seal.

The base preferably has a support for supporting an aerosol or gas delivery head. Also, there is preferably a shell configured to form an enclosure together with the base. There may be an extraction port for attachment of an extraction system to extract gas from said volume in use.

In some examples, the base is annular, configured to fully surround the patient's mouth and nose. In some examples, the base comprises a spine on which there is an inner soft layer for engaging the patient's face. In some examples, the support is mounted to the spine,

In some examples, the support extends across the base at or adjacent a central location to bisect the base. In some examples, the support comprises openings to receive nasal prongs of an aerosol delivery head. In some examples, the shell has at least one opening for passage of an aerosol delivery tube. In some examples, the shell comprises a pair of openings to allow connection of an aerosol head at either side. In some examples, the shell comprises blanks to seal off an un-used opening. In some examples, the shell is configured to snap-fit to the base.

Preferably, the extraction port is located at a location approximately central to the base for alignment in use with a patient's mouth. Preferably, the interface further comprises a pressure sensor. In some examples, the pressure sensor is mounted to the shell. In some examples, the shell includes at least one vent.

We also describe an aerosol treatment system comprising a patient interface to cover a patient's mouth and nose, and a high flow treatment system linked with the mask. In some examples, the system further comprises an aerosol delivery apparatus, an extraction apparatus, and a controller configured to control delivery of aerosol and/or gas to the interface and to extract gases from a volume enclosed by the interface. In some examples, the high flow treatment system is a high flow nasal treatment system (HFNT).

In some examples, the system includes sensors for detecting patient breathing and the controller is configured to provide breath-synchronised delivery. In some examples, the system comprises a heater and a humidifier, separately or combined, to provide a heated humidified air/O₂ mixture delivered to the interface.

Preferably, the system comprises a valve arranged to split delivery flow into an aerosol branch with a nebulizer and a parallel bypass branch, and the branches merge into a common tube which leads to the interface.

Preferably, the controller is configured to dynamically control the system to provide scenarios including some or all of:
a full bypass;
no bypass with continuous aerosol delivery;
no bypass with breath synchronised aerosol delivery according to signals from a pressure sensor in the volume enclosed by the interface; and/or
breath-synchronised bypass with divert through the bypass during exhalation;
continuously-on aerosol generation allowing aerosol to accumulate in a chamber and during inhalation divert the flow through the chamber for increased aerosol concentration.

Preferably, the aerosol generator includes a chamber having an increased volume to slow down the delivery flowrate. Preferably, the controller is configured to provide step-down aerosol delivery. Preferably, the controller is configured to reduced gas flowrate and increased aerosol delivery during inhalation to improve dose efficiently.

Preferably, the controller is configured to provide a period of reduced flowrate which is short enough to prevent de-recruitment effects. Preferably, the controller is configured to provide dynamic extraction according to monitoring of pressure in the interface volume.

Preferably, the controller is configured to adapt a baseline extraction to match a high flow therapy setting, and to dynamically change an extraction rate to match the patient's breath pattern.

Preferably, the extraction apparatus includes a filter. Preferably, the filter is adapted to capture pathogens or drug before venting to ambient.

Preferably, the controller is configured to increase power supplied to an extraction source to keep the extraction flowrate consistent over time as the filter approaches saturation.

Preferably, the extraction apparatus comprises a condenser to take vapour out of extracted gas, preferably prior to it reaching a filter. Preferably, the condenser is included in a heat pump in which heat collected is used to heat delivery flow to the patient.

The aerosol treatment system may include an interface of any example described above.

### DETAILED DESCRIPTION OF THE INVENTION

The invention will be more clearly understood from the following description of some embodiments thereof, given by way of example only with reference to the accompanying drawings in which:

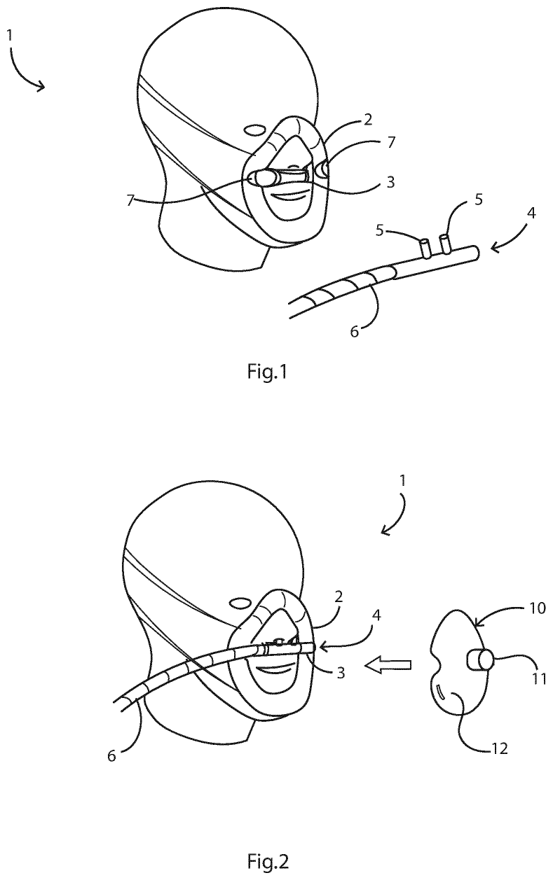

FIGS. 1 to 4 are perspective views of an aerosol high flow mask being applied in four steps, respectively;

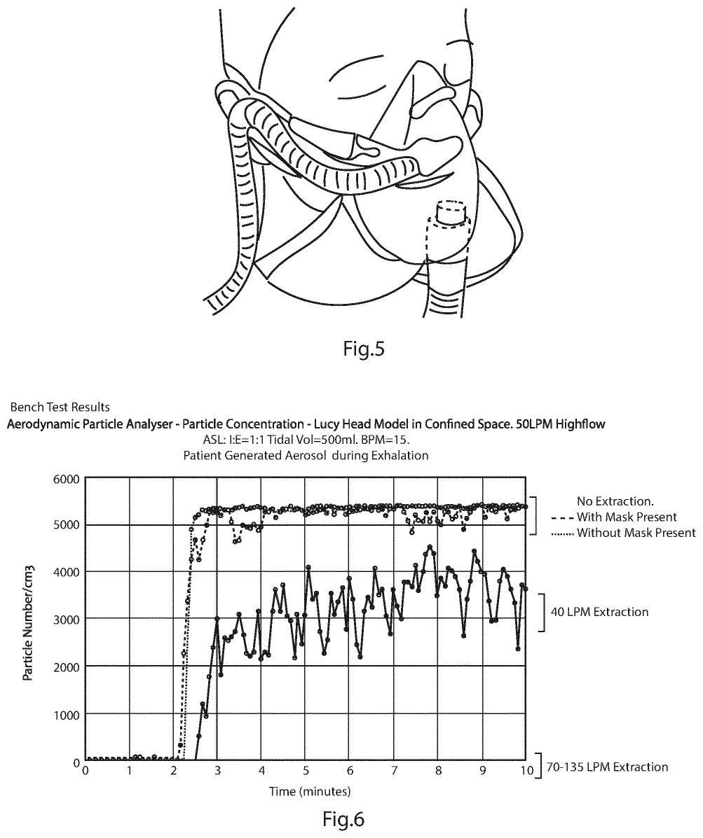

FIG. 5 is a diagram of a mask in position on a demonstration patient head model used for testing;

FIG. 6 is a plot of bench test results showing particle numbers vs. time determined by an aerodynamic particle analyser for the set-up of FIG. 5 and 50 LPM High Flow therapy, and

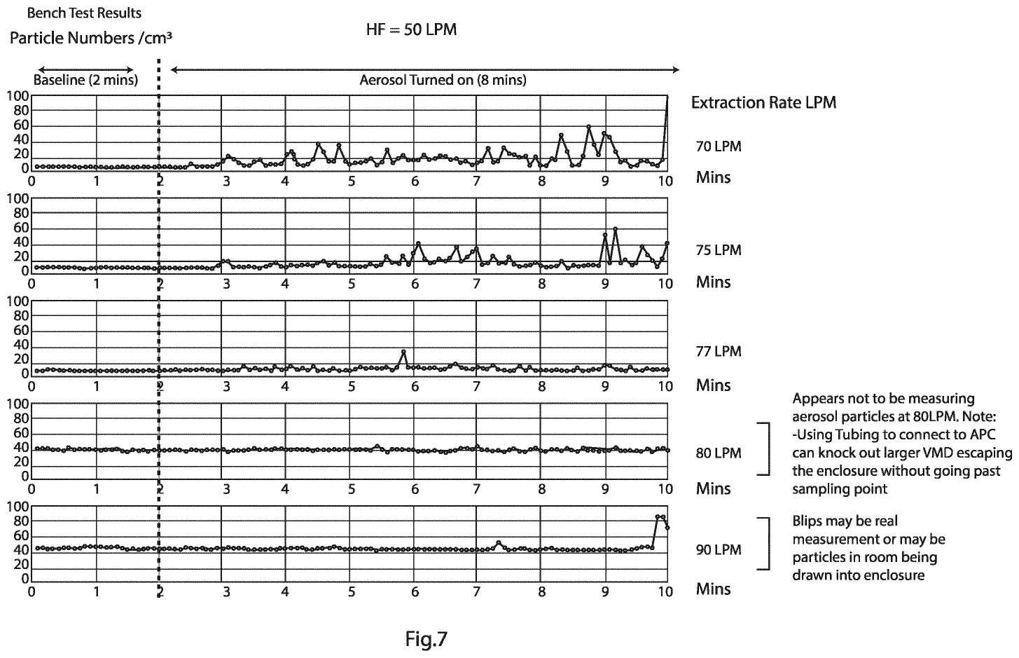

FIG. 7 is a plot of bench test results showing particle measurements with time for various high flow rates, also for the set-up of FIG. 5 and in this case showing in more detail the plots for the higher-level extraction of 70 to 135 LPM;

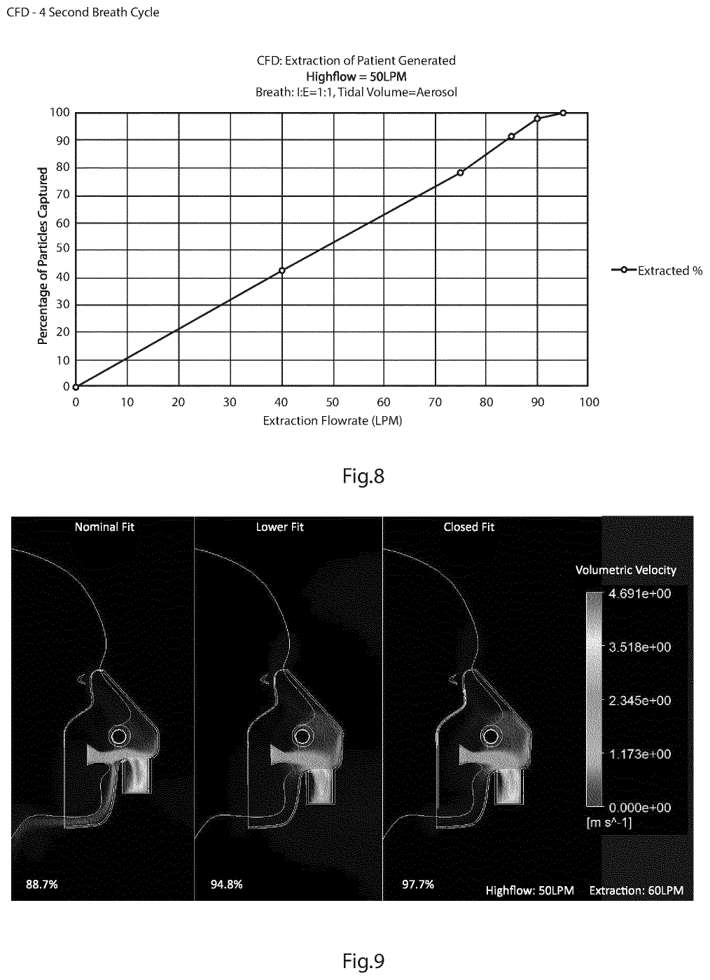

FIG. 8 is a plot, also for this set-up, showing percentage of particles captured with extraction flow rate;

FIG. 9 shows fluid flows detected by imaging sensors, coded to illustrate volumetric velocity within the mask for nominal mask fit, lower mask fit, and closed mask fit;

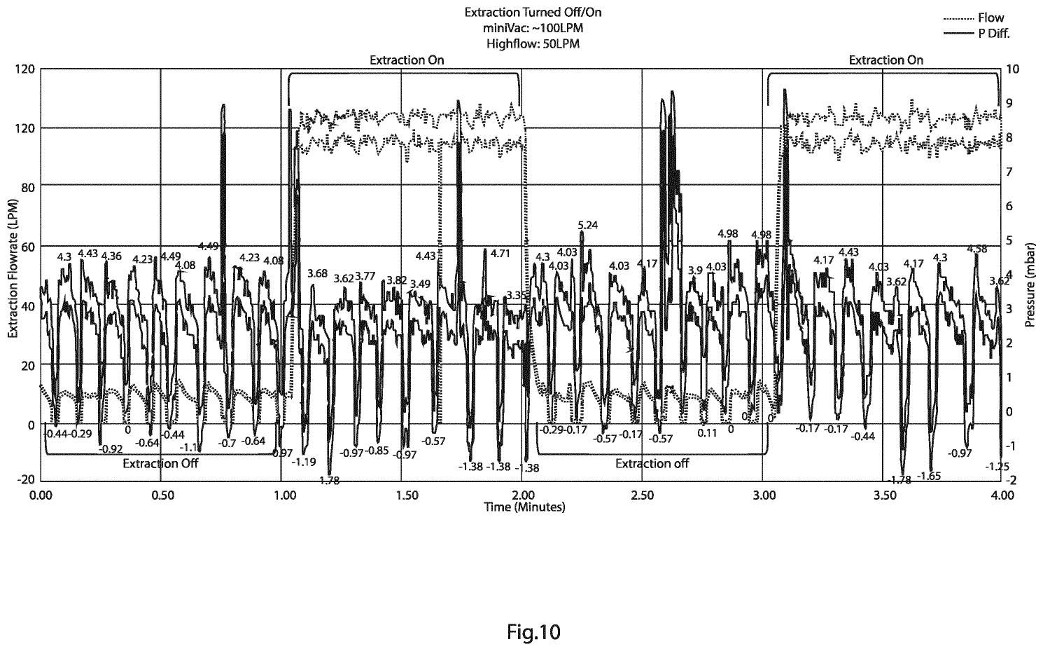

FIG. 10 is a set of plots of extraction flowrate with time for a set-up as shown in FIG. 5 and with a nasal pressure sensor added;

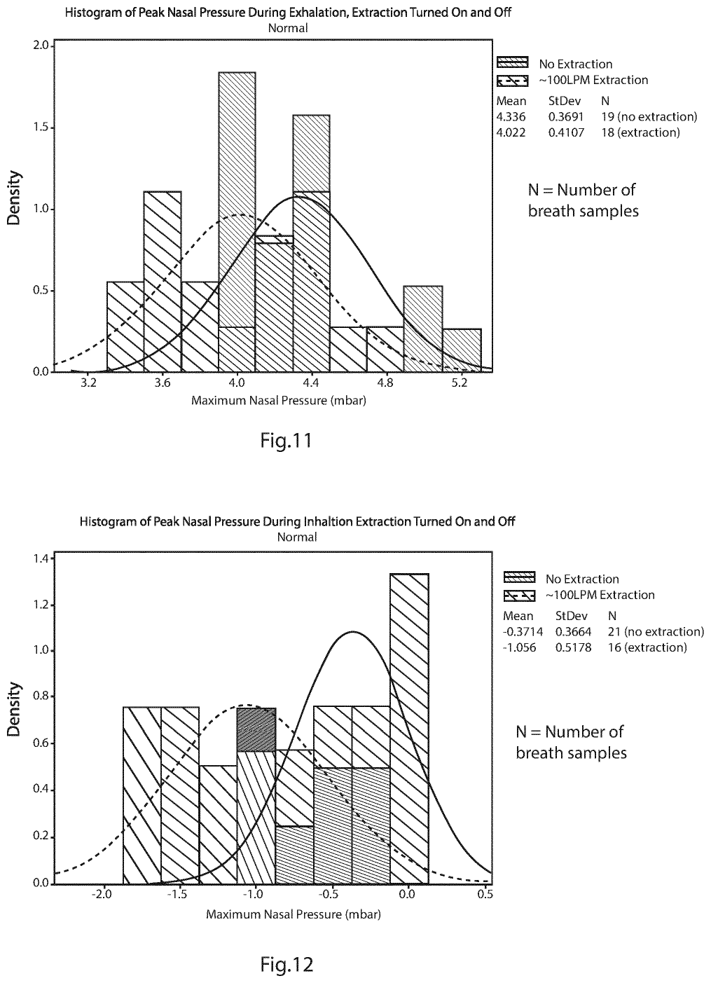

FIG. 11 is a histogram of peak nasal pressure for extraction being turned on and off, and FIG. 12 is a histogram of minimum nasal pressure for extraction turned on and off;

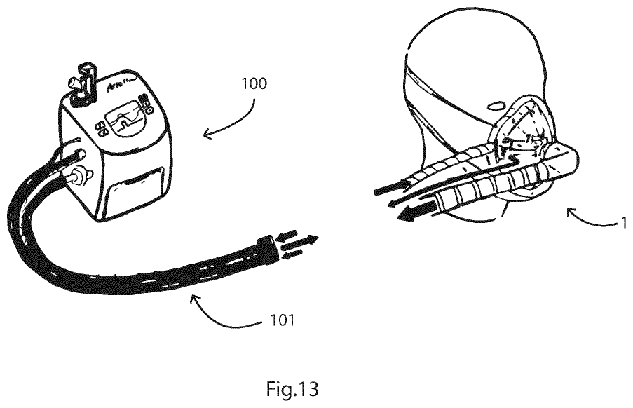

FIG. 13 is a diagram showing the mask of FIGS. 1 to 4 in place, together with a controller;

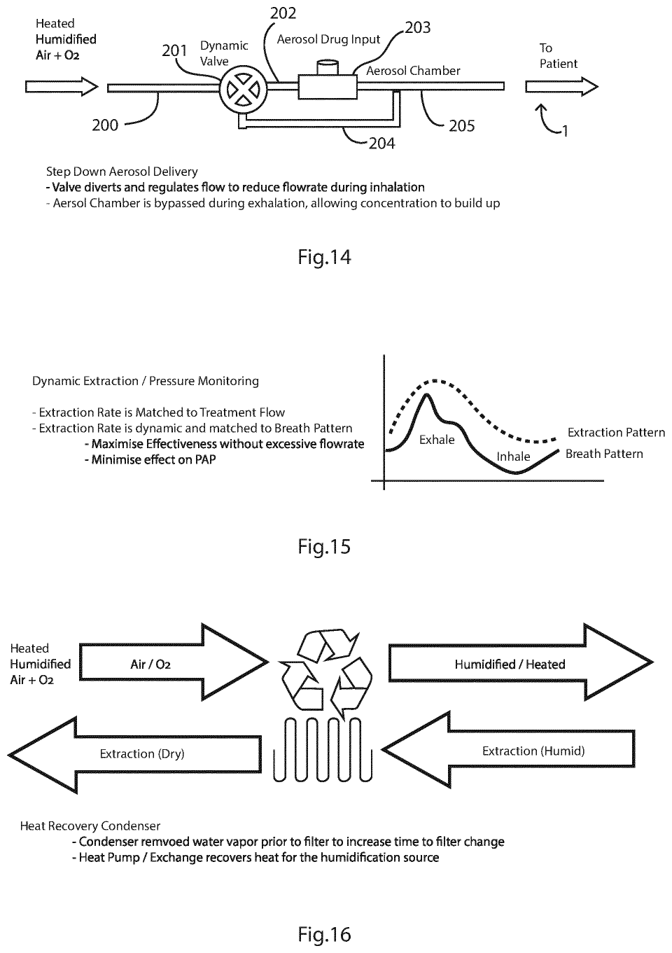

FIG. 14 is a flow diagram showing how reduced flow is achieved during inhalation and the aerosol chamber limb is bypassed during inhalation;

FIG. 15 is a pair of plots showing dynamic extraction and pressure monitoring, showing relationship between extraction and breath flows, in which extraction is matched to aerosol flow; and

FIG. 16 is a diagram showing flows of humidified and heated air and O₂ towards the mask and extracted humid breath which is dried in the extraction direction by virtue of a heat recovery condenser and a heat exchanger which recovers heat for humidification.

### DESCRIPTION OF THE EMBODIMENTS

Referring to FIG. 1 a patient interface 1 comprises an annular soft seal base 2 configured to fit around a patient's mouth and nose with a large contact surface of resilient material which is suited to skin contact. The base 2 has a relatively rigid core surrounded on at least the patient side with soft material for patient facial contact. The base spine supports a prong receiver 3 extending across the base 2, and being of a plastics material having a rugged strength to support attachment of a nasal prong head 4 with prongs 4 at the end of tubing 6 for delivery of high flow gas and aerosol to the nostrils.

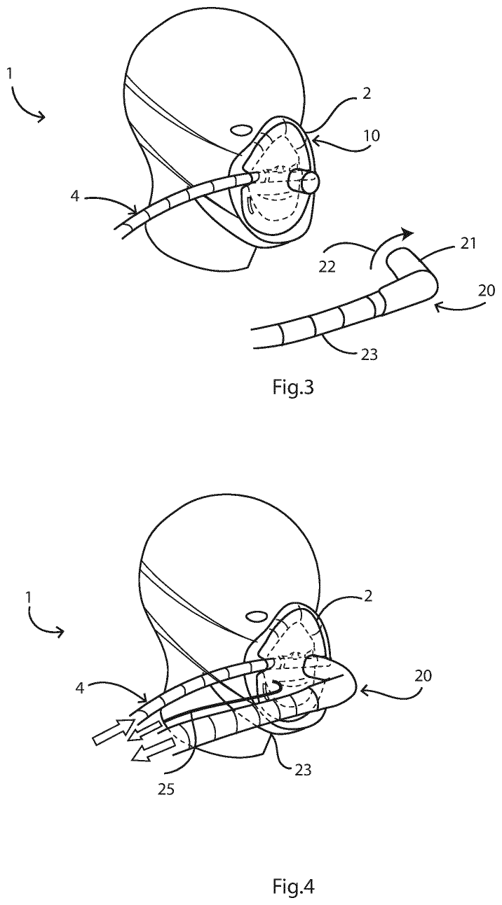

FIG. 2 shows that the interface also includes a shell 10 which fits with a seal to the base 2 and the support 3 to form an enclosed volume around the patient's mouth and nasal area. FIG. 3 shows an extraction head 20 which has an L-shaped conduit 21 with a flange 22 configured to fit to a cylindrical port 11 of the shell.

FIG. 4 shows the full interface 1, with all of these components in place, together with a lead 25 from a pressure sensor within the volume formed by the base 2 and the shell 10. The pressure sensor is attached to the inside surface of the shell 10.

The patient interface is modular, the soft seal base 2 attaching to the patient's head with securing head straps 8. The support 3 supports the prong head 4 with the prongs 5 correctly aligned. It is envisaged that in other embodiments the prong support is self-supporting by way of head straps rather than being attached to the soft seal base, especially for uses without extraction.

The soft seal base 2 is placed on the face first and a comfortable sealing surface is established. This establishes a gas-tight seal and provides a support mechanism for the high flow therapy tubing, and the clinician can conveniently and accurately set up the patient's nasal prongs 3 secured to the soft seal base 2. The fit of the prongs 5 can be checked and adjusted.

The shell 10 can now be assembled. The perimeter of the shell 10 interfaces with the base such that it self-locates and forms a seal, the soft seal base 2 providing a means of securing the shell 10 in place by for example elastic straps, hook-and-loop fasteners, or clips. It is preferred that the shell fit by way of a snap-fit connection with resilient edges. The shell 10, nasal prongs 4/5, and the soft seal base 2 are profiled such that the high flow therapy tubing and head 4 can enter from either the left or the right, and a seal is still established without the need for an additional part. In another embodiment an additional capping feature can be provided.

The shell 10 preferably includes one or more vents to prevent an excessive negative pressure drop within the volume formed by the base 2 and the shell 10 due to extraction. These are located away from exhalation/exhaust airstreams of the mouth and the nostrils. The shell 10 has a port 11 to attach a means of suction, in this case the extraction head 20. The position of the extraction port 11 is such that in use it is opposed to the mouth and nostrils for optimal collection of exhaled/exhausted gasses and particles. The vent or vents may not be in the mask itself, and could for example be part of an exhalation tube. The benefit of a vent is that, because the mask is very effective at sealing the space around the nose and mouth, the operation of the high flow system and the forced extraction system does not cause the system to be too intrusive by acting effectively akin to a ventilator, in which all inhalation and exhalation is controlled. There may for example be a very soft opening on an inhale valve to not affect breathing, and/or a pressure release valve for safely in case of reduced extraction. The vent may have a suitable filter to block outflow of unwanted droplets to avoid contamination of the environment.

The shell also has a retainer to attach a sensor for measuring the internal mask pressure.

FIGS. 6 to 8 shows test results for ambient aerosol particles in the environment surrounding the patient's head for with the mask and system shown in FIG. 5. This mask is conventional in terms of how it is configured to engage the face, simply a flexible clear polymer with holes cut to take the HFNT delivery and extraction hoses. It does not have the benefits of the mask of FIGS. 1 to 4.

FIG. 6 demonstrates an extraction flowrate in the region of 80 LPM to 100 LPM is required to capture approximately all aerosol particles, in these examples with treatment at 50 LPM. FIG. 6 demonstrates the major differences between the bands of 7- to 135 LPM, 40 LPM, and no extraction. In the latter there is essentially no difference between situations where the mask is present and is not, because of losses around the edges of the mask. FIG. 7 shows more detail for the higher extraction rate band, with the vertical axes showing particles in the tens per cm³. FIG. 9 is a summary histogram showing that there is a liner relationship between extraction flowrate and percentage of particles captured, reaching full capture at about 90 LPM extraction.

The positioning of the extraction port opposite the patient's mouth affects the rate of emission capture for a given extraction rate. In FIG. 9, the ‘Lower Fit’ outperforms the ‘Nominal Fit’ in terms of capture of emissions (94.8% vs. 88.7%). The degree of sealing between the mask and the face affects rate of emission capture for a given extraction rate. In FIG. 9 ‘Closed Fit’ is the same as ‘Lower Fit’ except that there is sealing at the cheeks. At the same level of extraction (60 LPM) versus the same emission flowrate (50 LPM, there is no breath in this example) the ‘Closed Fit’ outperforms the ‘Lower Fit’ in term of capture of emissions (97.7% vs. 94.8%).

The effect of extraction on the nasal cavity has been investigated. A test setup as shown in FIG. 5 but with addition of a nasal pressure sensor involved:
Delivery of humified high flow @ 50 LPM
Pressure tube inserted into nasal cavity
Extraction flowrate either Off or @100 LPM
Flowmeter in-line with extraction source.

The results of this are illustrated in FIGS. 10 to 12, which displays the distribution of the nasal pressure peaks, which occur during exhalation, with and without extraction. The average difference is a reduction of 0.3 mBar when extraction is applied; the difference of the maximums is 0.53 mBar.

FIG. 12 displays the distribution of the nasal pressure troughs (these occur during inhalation) with and without extraction. The average difference is a reduction of 0.68 mBar when extraction is applied; the difference of the minimums is also 0.68 mBar.

These results demonstrate the advantages of decreasing extraction during exhalation and not having any extraction during inhalation.

Major advantages of the invention include:
Flexible patient-engaging side of the base 2 provides consistent sealing, especially allowing successful extraction at lower flow rates.
A predictable seal allows less safety factor buffer on the applied extraction rate.
Less extraction allows less pressure drop, less noise, more extraction source options, controlled vent ports.
Maximise extraction for a given extraction flowrate.
Avoidance of occlusion with patient's face.
Reduced wind noise/wind feeling
Sized to alleviate pressure drop by a predictable quantity
Modularity, allowing fitting of the base to be secured to head and adjusted for sealing and comfort independent of the therapy.
Allows clinical access to setup/check/change prongs.
Pressure Port.
Provides means of measuring pressure internal to the mask.
Can be used as a safety measure.
Can be used to detect the breath and dynamically control the extraction rate.
Allows for more effective extraction with reduced impact on pressure drop.

The patient interface 1 can connect to a standalone aerosol/high flow therapy device 100 by a tubing set 101 as depicted in FIG. 13. This allows breath-synchronised step-down aerosol delivery. As illustrated in FIG. 14 a heated humidified air/O₂ mixture is delivered on a tube 200, and flow is split by a valve 201 into an aerosol branch 202 with a nebulizer 203 and a parallel bypass branch 204. The branches merge into a common tube 205 which leads to the interface 1.

In some examples, the aerosol delivery path can include an aerosol chamber having an increased volume to slow down the flowrate at the point of aerosol delivery

### Bypass Scenarios

The valve 201 can dynamically throttle the flowrate, and can dynamically divert the flowrate to provide scenarios such as:
Full bypass, normal high flow treatment with no aerosol.
No bypass, open throttle, continuous aerosol delivery.
No bypass, open throttle, breath synchronised aerosol delivery (pressure sensor used to detect breath pattern).
Breath-synchronised bypass: divert through bypass during exhalation. Nebuliser is continuously on allowing aerosol to accumulate in the chamber. During inhalation divert the flow through the chamber for increased aerosol concentration.

### Step-Down Delivery

Step-down aerosol delivery: when switching to the aerosol path 202 during inhalation, the average flowrate is reduced. This will improve dose efficiently. The period of reduced flowrate is short to prevent de-recruitment effects. There is a ramp down/ramp up of the reduced flowrate, and these ramps can be controlled/modulated to minimise de-recruitment and discomfort.

### Dynamic Extraction/Pressure Monitoring

The system controller can adapt the baseline extraction to match the high flow therapy setting. The controller can be programmed to dynamically change the extraction rate to match the breath pattern, as illustrated in FIG. 15. This can maximise the effectiveness of extraction, and therefore reduce the required extraction rate. This has the benefit of reducing the impact on the treatment pressure, and also reduces the extraction source requirements.

### Filtration

An advantageous part of the extraction system is that there is filtration in line with the extracted airflow to capture any pathogens or drug before it is vented to the ambient room. This can be a standard commercial filter that can be changed out by the clinicians. Due to the large levels of humidity in the expelled gas, the filter will become saturated, and the filter regime adapted accordingly. The system can determine the actual flowrate based off the mask pressure readings. Or, additional flow and pressure sensors could be employed on the system side of the filter. The system can increase the power supplied to the extraction source to keep the extraction flowrate consistent over time as the filter approaches saturation.

### Condenser

A condenser can be employed to take vapour out of the extracted gas prior to it reaching the filter. This can prolong the life of the filter. The condensing mechanism is preferably such that the surfaces that make contact with the extracted gasses are part of a disposable circuit. A heat pump (for example using a Peltier heat exchanger) can be employed to increase the rate of condensing, as illustrated in FIG. 16. The heat collected in this exchange can be employed in heating of the high flow therapy delivered to the patient. This would have energy saving benefits.
No Fugitive emissions of treatment drug
No spreading of patient pathogens
Increased drug efficiency
Increased drug rate

The invention is not limited to the embodiments described but may be varied in construction and detail. For example, it is envisaged that the mask is provided as a pre-assembled component, perhaps using a sizing chart to allow a clinician to preconfigure and position nasal prongs. The mask, if it does not have a removable shell may have an access flap to allow adjustment of the nasal prongs. The performance features and advantages described for an interface having conventional features apply to an interface of FIGS. 1 to 4, and it is expected that performance would be better because of the enhanced sealing and other advantages described with reference to FIGS. 1 to 4. Irrespective of the interface used, any interface which supports the HFNT delivery and extraction and provides a closed or near-closed environment around the mouth and nose is advantageous as described above with reference to FIGS. 5 to 12 and 14 to 16, and where the interface of FIGS. 1 to 4 and 13 is used it enhances the closed nature of this volume around the nose and mouth.

1-33. (canceled)

34. A patient interface for aerosol treatment, the interface comprising a base configured to form a volume surrounding at least part of a patient's mouth and nose and engage a patient's skin with a resilient seal, a support on the base for supporting an aerosol or gas delivery head, a shell configured to form an enclosure together with the base, and an extraction port for attachment of an extraction system to extract gas from the volume in use.

35. The interface as claimed in claim 34, wherein the base is annular, configured to fully surround the patient's mouth and nose, and wherein the base comprises a spine on which there is an inner soft layer for engaging a face of the patient, and wherein the support is mounted to the spine.

36. The interface as claimed in claim 34, wherein the support extends across the base at or adjacent a central location to bisect the base, and wherein the support comprises openings to receive nasal prongs of an aerosol delivery head, and wherein the shell has at least one opening for passage of an aerosol delivery tube.

37. The interface as claimed in claim 34, wherein the shell has at least one opening for passage of an aerosol delivery tube, and wherein the shell comprises a pair of openings to allow connection of an aerosol head at either side, and wherein the shell comprises blanks to seal off an un-used opening.

38. The interface as claimed in claim 34, wherein the shell is configured to snap-fit to the base.

39. The interface as claimed in claim 34, wherein the extraction port is located at a location approximately central to the base for alignment in use with a patient's mouth.

40. The interface as claimed in claim 34, further comprising a pressure sensor mounted to the shell.

41. The interface as claimed in claim 34, wherein the shell includes at least one vent.

42. An aerosol treatment system comprising a patient interface to cover a patient's mouth and nose, a high flow treatment system, and a controller, and further comprising an aerosol delivery apparatus, an extraction apparatus, and a controller configured to control delivery of aerosol and/or gas to the interface and to extract gases from a volume enclosed by the interface.

43. The aerosol treatment system as claimed in claim 42, wherein the high flow treatment system is a high flow nasal treatment system (HFNT).

44. The aerosol treatment system as claimed in claim 42, wherein the system includes sensors for detecting patient breathing and the controller is configured to provide breath-synchronized delivery.

45. The aerosol treatment system as claimed in claim 42, wherein the system comprises a heater and a humidifier, separately or combined, to provide a heated humidified air/O2 mixture delivered to the interface.

46. The aerosol treatment system as claimed in claim 42, wherein the system comprises a heater and a humidifier, separately or combined, to provide a heated humidified air/O2 mixture delivered to the interface, and wherein the system comprises a valve arranged to split delivery flow into an aerosol branch with a nebulizer and a parallel bypass branch, and the branches merge into a common tube which leads to the interface.

47. The aerosol treatment system as claimed in claim 42, wherein the system comprises a heater and a humidifier, separately or combined, to provide a heated humidified air/O2 mixture delivered to the interface, and wherein the system comprises a valve arranged to split delivery flow into an aerosol branch with a nebulizer and a parallel bypass branch, and the branches merge into a common tube which leads to the interface wherein the controller is configured to dynamically control the system to provide scenarios including one or more of:
a full bypass;
no bypass with continuous aerosol delivery;
no bypass with breath synchronised aerosol delivery according to signals from a pressure sensor in the volume enclosed by the interface; and/or
breath-synchronised bypass with divert through the bypass during exhalation; continuously-on aerosol generation allowing aerosol to accumulate in a chamber and during inhalation divert the flow through the chamber for increased aerosol concentration.

48. The aerosol treatment system as claimed in claim 42, further including an aerosol generation, the aerosol generator including a chamber having an increased volume to slow down a delivery flowrate.

49. The aerosol treatment system as claimed in claim 42, wherein the controller is configured to provide step-down aerosol delivery, and wherein the controller is configured to reduced gas flowrate and increased aerosol delivery during inhalation to improve dose efficiently, and wherein the controller is configured to provide a period of reduced flowrate which is short enough to prevent de-recruitment effects.

50. The aerosol treatment system as claimed in claim 42, wherein the controller is configured to provide dynamic extraction according to monitoring of pressure in the interface volume, and wherein the controller is configured to adapt a baseline extraction to match a high flow therapy setting, and to dynamically change an extraction rate to match a breath pattern of the patient.

51. The aerosol treatment system as claimed in claim 42, wherein the extraction apparatus includes a filter, and wherein the extraction apparatus includes a filter, and wherein the filter is adapted to capture pathogens or drug before venting to ambient.

52. The aerosol treatment system as claimed in claim 51, wherein the controller is configured to increase power supplied to an extraction source to keep an extraction flowrate consistent over time as the filter approaches saturation.

53. The aerosol treatment system as claimed in claim 42, wherein the extraction apparatus comprises a condenser to take vapour out of extracted gas prior to it reaching a filter and wherein the condenser is included in a heat pump in which heat collected is used to heat delivery flow to the patient.

54. The aerosol treatment system as claimed in claim 42, wherein the interface comprises a base configured to surround at least part of a patient's mouth and nose and engage a patient's skin with a resilient seal, a support on the base for supporting an aerosol or gas delivery head, a shell configured to form an enclosure together with the base, and an extraction port for attachment of an extraction system to extract gas from said volume in use.
